(19) United States
(12) Patent Application Publication
(10) Pub. No.: US 2004/0087531 A1
(43) Pub. Date: May 6, 2004

(54) Compositions and methods for the treatment of cancer

(76) Inventors: Adam Telerman, Paris (FR); Robert Amson, Paris (FR); Marius Tuijnder, Mechelen (BE); Laurent Susini, Montmorency (FR)

Correspondence Address: JONES DAY, 51 Louisiana Aveue, N.W, WASHINGTON, DC, 20001-2113, US

(21) Appl. No.: 10/439,248

(22) Filed: May 16, 2003

Related U.S. Application Data
(60) Provisional application No. 60/378,092, filed on May 16, 2002.

(51) Int. Cl. A61K 48/00

(52) U.S. Cl. 514/44

(57) ABSTRACT

Composition and methods of treating, preventing, and managing cancer by inhibiting the expression of the gene tpt1 are disclosed. In addition, a method of identifying genes that are involved in the tumor reversion of two or more types of cancers is also disclosed.

This application claims the benefit of U.S. Provisional Application No. 60/378,092, filed May 16, 2002, the entirety of which is incorporated herein by reference.

### 1. FIELD OF THE INVENTION

This invention relates to methods of treating and managing cancer using compounds that modulate the synthesis or expression of the gene tpt1. The invention further relates to methods of identifying genes involved in tumor reversion.

### 2. BACKGROUND OF THE INVENTION

The incidence of cancer continues to climb as the general population ages, as new cancers develop, and as susceptible populations (e.g., people infected with AIDS) grow. A tremendous demand therefore exists for new methods and compositions that can be used to treat patients with cancer.

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multi-step process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (third ed., Mosby, St. Louis: 1993).

Although treatments for various cancers are known in the art, it is still difficult—if not impossible—to predict ab initio the effect a particular combination of drugs may have on a given form of cancer. Most cancer research is primarily focused on understanding how normal cells become malignant and on what the genomic alterations underlying tumor formations are. To that end, much research has been directed to understanding the consequences of gene expression variations by making comparisons between tumor cells and their normal counterparts. These normal counterparts, however, can only provide limited information as to how tumor cells learn to quit the malignant characteristics associated with them.

Tumor reversion is a spontaneous process wherein malignant cells revert to more normal phenotypes. The study of tumor reversion has identified genes that are differentially expressed between tumor cells and their revertants. See Tuynder et al., Proc. Natl. Acad. Sci. USA 99(23): 14976-14981 (2002). One of those is the gene tpt1, which produces the Translationally Controlled Tumor Protein (“TCTP”). Because tpt1/TCTP was identified as the human histamine releasing factor, research concerning it has focused on its role in allergic response. See, e.g., McDonald et al., Science 269: 688-690 (1995) It has also been shown that TCTP is one of the first proteins to be induced in Ehrlich ascites tumor cells after mitotic stimulation. Bohm et al., Biochem. Int. 19: 277-286 (1989). In addition, tpt1/TCTP has been described as binding a Bcl-2 homologue in yeast two-hybrid assay, and identified as an antiapoptotic protein. Li et al., J BioL Chem. 276: 47542-47549 (2001).

### 3. SUMMARY OF THE INVENTION

This invention is based, in part, on the discovery that tumor reversion can be effected by controlling the synthesis or expression of tpt1. Thus, this invention is generally related to a method of suppressing growth of a cancer cell using a compound that modulates the synthesis or expression of the gene tpt1. Specific methods of the invention induce apoptosis of the cancer cell or induce its reversion to a cell that exhibits normal phenotype.

This invention further encompasses a method of treating or managing cancer by administering to a patient in need thereof a compound that modulates the synthesis or expression of the gene tpt1. Also encompassed by this invention are pharmaceutical compositions and single unit dosage forms comprising a compound that modulates the synthesis or expression of the gene tpt1.

Further embodiment of this invention includes a method of identifying genes that are differentially expressed in two or more tumor cell/revertant pairs. This provides a method of identifying an agent that may be universally effective against various type of cancer cells.

3.1 Definitions

As used herein, and unless otherwise indicated, the term “suppression” or “suppressing”, when used in relation to the growth of a cell, means retardation or prevention of the growth of the cell. Such suppression may be, but is not necessarily, accoplished through mechanisms such as, but not limited to, tumor reversion and cell apoptosis. In specific embodiments of this invention, growth of a cell is suppressed when the growth is slowed by greater than about 20, 30, 50, 75, 100 or 200 percent as determined by, e.g., mass tumor volume.

As used herein, and unless otherwise indicated, the term “inhibiting the synthesis or expression” of a gene means impeding, slowing or preventing one or more steps by which the end-product protein encoded by said gene is synthesized. Typically, the inhibition involves blocking of one or more steps in the gene's replication, transcription, splicing or translation through a mechanism that comprises a recognition of a target site located within the gene sequence based on sequence complementation. In a specific embodiment, inhibition of tpt1 reduces the amount of TCTP in the cancer cell by greater than about 20, 50, or 70 percent. The amount of TCTP can be determined by well-known methods including, but are not limited to, densitometer, fluorometer, radiography, luminometer, antibody-based methods and activity measurements.

As used herein, and unless otherwise indicated, the term “antisense oligonucleotide” refers to an oligonucleotide having a sequence complementary to a target DNA or RNA sequence.

As used herein, and unless otherwise indicated, the term “part,” as used to designate a portion of a DNA or RNA, means a portion of at least 15, 20, or 25 nucleotides.

As used herein, and unless otherwise indicated, the term “tpt1 siRNA” denotes a small interfering RNA that has a sequence complementary to a sequence within the tpt1 gene. Typically, siRNAs are about 20 to 23 nucleotides in length.

As used herein, and unless otherwise indicated, the term “complementary,” when used to describe a sequence in relation to a target sequence, means that the sequence is able to bind to the target sequence in a cellular environment in a manner sufficient to disrupt the function (e.g., replication, splicing, transcription or translation) of the gene comprising the target sequence. The binding may result from interactions such as, but not limited to, nucleotide base parings (e.g., A-T/G-C).

In particular embodiments of the invention, a sequence is complementary when it hybridizes to its target sequence under high stringency, i.e., conditions for hybridization and washing under which nucleotide sequences, which are at least 60 percent (preferably greater than about 70, 80, or 90 percent) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which is incorporated herein by reference. Another example of stringent hybridization conditions is hybridization of the nucleotide sequences in 6× sodium chloride/sodium citrate (SSC) at about 45° C., followed by 0.2×SSC, 0.1% SDS at 50-65° C. Particularly preferred stringency conditions are hybridization in 6× sodium chloride/sodium citrate (SSC) at about 45C, followed by one or more washes in 0.2×SSC, 0.1% SDS at 50C. Another example of stringent hybridization conditions are hybridization in 6× sodium chloride/sodium citrate (SSC) at about 45C, followed by one or more washes in 0.2×SSC, 0.1% SDS at 55C. A further example of stringent hybridization conditions are hybridization in 6×sodium chloride/sodium citrate (SSC) at about 45C, followed by one or more washes in 0.2×SSC, 0.1% SDS at 60C. Preferably, stringent hybridization conditions are hybridization in 6× sodium chloride/sodium citrate (SSC) at about 45C, followed by one or more washes in 0.2×SSC, 0.1% SDS at 65C. Another preferred example of stringent hybridization condition is 0.5M sodium phosphate, 7% SDS at 65C, followed by one or more washes at 0.2×SSC, 1% SDS at 65C.

Depending on the conditions under which binding sufficient to disrupt the functions of a gene occurs, a sequence complementary to a target sequence within the gene need not be 100 percent identical to the target sequence. For example, a sequence can be complementary to its target sequence when at least about 70, 80, 90, or 95 percent of its nucleotides bind via matched base pairings with nucleotides of the target sequence.

When used to describe the sequences of siRNAs, the term “corresponding to,” as used herein, means that an siRNA has a sequence that is identical or complementary to the portion of target mRNA that is transcribed from the denoted DNA sequence.

### 4. BRIEF DESCRIPTION OF FIGURES

FIG. 1A illustrates the number and size of the colonies of tumor cell lines K562, BT20, T47D and MCF7 and their revertants or SIAH-1 transfected counterparts, as measured by an in vitro soft agar assay;

FIG. 1B illustrates the comparison of tumorigenicity between cell lines K562, U937, BT20 and MB231 and their revertants, as measured in vivo in scid/scid mice;

FIG. 1C illustrates the results of PCR analysis specific for a 254 base pair regions of H-1 parvovirus in tumor cells and their revertants;

FIG. 2 illustrates a schematic diagram of identifying genes commonly involved in tumor reversion in various cell lines using the differential expression analysis in various tumor cell/revertant or tumor cell/SIAH-1 transfected counterpart pairs;

FIG. 3 illustrates the results from differential expression analysis in various cell lines, wherein two hundred sixty three genes that are differentially expressed between a tumor cell and a revertant are identified;

FIG. 4A illustrates a northern blot analysis of tpt1 in U937/US4.2, U937/U937-SIAH-1 and MCF7/MCF7-SIAH-1 cell lines;

FIG. 4B illustrates a western blot analysis of TCTP in U937/US4.2, U937/U937-SIAH-1 and MCF7/MCF7-SIAH-1 cell lines, and M1 and LTR6 stably transfected with temperature sensitive p53 val135 mutant;

FIG. 5A illustrates a western blot analysis of TCTP in U937 cells stably transfected with vector alone or the vector containing antisense tpt1 cDNA;

FIG. 5B illustrates a PARP cleavage analysis of U937 cells stably transfected with vector alone or the vector containing antisense tpt1 cDNA;

FIG. 5C illustrates the content of annexin V in U937 cells stably transfected with vector alone or the vector containing antisense tpt1 cDNA;

FIG. 5D illustrates the results obtained from terminal deoxynucleotidyltransferase-mediated dUTP end labeling (TUNEL) assay in U937 cells stably transfected with vector alone or the vector containing antisense tpt1 cDNA;

FIG. 6 illustrates results obtained from in vivo tumorigenicity assays obtained from injecting U937 cells, U937 cells stably transfected by antisense PS-1, U937 cells stably transfected by SIAH-1, and U937 cells stably transfected by antisense tpt1 cDNAs;

FIG. 7 illustrates the expression of TCTP in various tumor cells and their normal counterparts;

FIG. 8A illustrates a western blot analysis of TCTP expression in MCF7 and T47D cells and the same cells stably transfected with tpt1 siRNA; and

FIGS. 8B-8H respectively illustrate three-dimensional reconstituted basement membrane matrigel cultures of: 184B5 cells; MCF7 in standard growth medium; MCF cells stably transfected with SIAH-1 cDNA; MCF cells transfected with trt siRNA; MCF7 cells transfected with tpt1 siRNA; T47D cells transfected with trt siRNA; and T47D cells transfected with tpt1 siRNA.

### 5. DETAILED DESCRIPTION OF THE INVENTION

This invention is generally related to treatment and management of cancer by inhibiting the expression of tpt1, which was discovered to be involved in the process of tumor reversion. Therefore, one embodiment of this invention is directed to a methods of suppressing the growth of a cancer cell, comprising contacting the cell with a compound that inhibits the synthesis or expression of tpt1 gene in an amount sufficient to cause such inhibition. Without being limited by theory, the inhibition is achieved through selectively targeting tpt1 DNA or mRNA, i.e., by impeding any steps in the replication, transcription, splicing or translation of the tpt1 gene. The sequence of tpt1 is disclosed in WO 02/64731 (SEQ. ID NO. 72), the entirety of which is incorporated herein by reference.

Further embodiments of this invention are directed to methods of suppressing growth of a cancer cell, comprising contacting the cell with a compound that has a sequence complementary to at least part of the tpt1 mRNA. In one embodiment, the compound is an oligonucleotide antisense to tpt1 mRNA. In a particular method, the oligonucleotide is a cDNA that transcribes into an RNA having a sequence complementary to tpt1 mRNA. In another method, the compound is a tpt1 siRNA. Suitable siRNAs include, but are not limited to, those having a sequence corresponding to SEQ. ID NO. 1 or SEQ. ID NO. 2. In another method, the production of TCTP is inhibited by greater than about 20, 50, or 70 percent. In yet another method, the inhibition induces apoptosis or reversion of the cancer cell.

Another embodiment of this invention encompasses a method of treating, preventing or managing cancer comprising administering to a patient in need of such treatment or management a therapeutically or prophylactically effective amount of a compound that inhibits the synthesis or expression of tpt1 gene. This invention also encompasses methods of treating, preventing or managing cancer comprising administering to a patient in need of such treatment or management a therapeutically or prophylactically effective amount of a compound that has a sequence complementary to at least part of the tpt1 mRNA. In a particular method, the compound is an oligonucleotide antisense to tpt1 mRNA. Specifically, the oligonucleotide is a cDNA that transcribes into an RNA having a sequence complementary to tpt1 mRNA. In another method, the compound is a tpt1 siRNA. Suitable siRNAs include, but are not limited to, those having a sequence corresponding to SEQ. ID NO. 1 or SEQ. ID NO. 2.

This invention also encompasses pharmaceutical compositions and single unit dosage form comprising a compound that inhibits the synthesis or expression of the tpt1 gene. This invention further encompasses pharmaceutical compositions and single unit dosage form comprising a compound that has a sequence complementary to at least part of the tpt1 mRNA. In a particular composition, the compound is an oligonucleotide antisense to tpt1 mRNA. Specifically, the oligonucleotide is a cDNA that transcribes into an RNA having a sequence complementary to tpt1 mRNA. In another composition, the compound is a tpt1 siRNA. Suitable siRNAs include, but are not limited to, those having a sequence corresponding to SEQ. ID NO. 1 or SEQ. ID NO. 2.

This invention also encompasses a method of identifying genes involved in tumor reversion comprising: 1) determining a first set of genes that are differentially expressed in a tumor cell as compared to its revertant or SIAH-1 transfected counterpart; 2) determining a second set of genes that are differentially expressed in a tumor cell of a different cell line as compared to its revertant or SIAH-1 transfected counterpart; and 3) identifying a gene that is common in both the first and second sets. This method is useful for identification of genes that are commonly involved in tumor reversion of two or more types of cancers.

5.1 Inhibition of tpt1 Expression

The expression of tpt1 can be inhibited using any well-known methods that target the tpt1 gene or its mRNA. These methods include, but are not limited to, the use of antisense oligonucleotides, ribozymes, nucleic acids molecules that promote triple helix formation, and siRNAs or co-repression of a target gene by introducing a homologous gene fragment into the cell that harbors the target gene. Preferred methods employ antisense oligonucleotides or siRNAs.

Known methods of inhibition by targeting a specific gene sequence generally require that the compound to be administered possess a sequence complementary to the target sequence. Although 100 percent sequence identity is preferred, it is not required in order to practice this invention. In specific embodiments, a compound has a sequence that has about 70, 80, or 90 percent or more identity to the target tpt1 sequence.

5.1.1 Antisense Oligonucleotides

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins.

Antisense oligonucleotides of this invention include oligonucleotides having modified sugar-phosphodiester backbones or other sugar linkages, which can provide stability against endonuclease attacks. This invention also encompasses antisense oligonucleotides that are covalently attached to an organic or other moiety that increase their affinity for a target nucleic acid sequence. Agents such as, but not limited to, intercalating agents, alkylating agents, and metal complexes can be also attached to the antisense oligonucleotides of this invention to modify their binding specificities.

A preferred antisense oligonucleotide is a cDNA that, when introduced into a cancer cell, transcribes into an RNA molecule having a sequence complementary to at least part of the tpt1 mRNA.

5.1.2 siRNAs

In another embodiment, the expression of tpt1 is inhibited by the use of an RNA interference technique referred to as RNAi. RNAi allows for the selective knockout of a target gene in a highly effective and specific manner. This technique involves introducing into a cell double-stranded RNA (dsRNA), having a sequence corresponding to the exon portion of the target gene. The dsRNA causes a rapid destruction of the target gene's mRNA. See, e.g., Hammond et al., Nature Rev Gen 2: 110-119 (2001); Sharp, Genes Dev 15: 485-490 (2001), both of which are incorporated herein by reference in their entireties.

Methods and procedures for successful use of RNAi technology are well-known in the art, and have been described in, for example, Waterhouse et al., Proc. Natl. Acad. Sci. USA 95(23): 13959-13964 (1998). The siRNAs of this invention encompass any siRNAs that can modulate the selective degradation of tpt1 mRNA.

The siRNAs of this invention include modifications to their sugar-phosphate backbone or nucleosides. These modifications can be tailored to promote selective genetic inhibition, while avoiding a general panic response reported to be generated by siRNA in some cells. Moreover, modifications can be introduced in the bases to protect siRNAs from the action of one or more endogenous enzymes.

The siRNAs of this invention can be enzymatically produced or totally or partially synthesized. Moreover, the siRNAs of this invention can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although 100 percent sequence identity between the siRNA and the target region is preferred, it is not required to practice this invention. siRNA molecules that contain some degree of modification in the sequence can also be adequately used for the purpose of this invention. Such modifications include, but are not limited to, mutations, deletions or insertions, whether spontaneously occurring or intentionally introduced. Specific examples of siRNAs that can be used to inhibit the expression of tpt1 are described in detail in Example 6.7.

5.1.3 Other Methods of Targeting tpt1 DNA or mRNA

Ribozymes are enzymatic RNA molecules capable of catalyzing the specific cleavage of RNA. The characteristics of ribozymes are well-known in the art. See, e.g., Rossi, Current Biology 4: 469-471 (1994), the entirety of which is incorporated herein by reference. Without being limited by theory, the mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage, which was disclosed in U.S. Pat. No. 5,093,246, the entirety of which is incorporated herein by reference. If the sequence of a target mRNA is known, a restriction enzyme-like ribozyme can be prepared using standard techniques.

The expression of the tpt1 gene can also be inhibited by using triple helix formation. Nucleic acid molecules to be used in triple helix formation for the inhibition of transcription should be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarily to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The expression of tpt1 can be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a DNA or mRNA can also be used in connection with this invention without departing from the gist of this invention.

5.2 Methods of Treatment, Management or Prevention

One embodiment of this invention is directed to a method of treating or managing cancer comprising administering to a patient in need of such treatment or management a therapeutically or prophylactically effective amount of a compound that inhibits the synthesis or expression of tpt1 gene.

As used herein, and unless otherwise indicated, the term “treating cancer” or “treatment of cancer” means to inhibit the replication of cancer cells, inhibit the spread of cancer, decrease tumor size, lessen or reduce the number of cancerous cells in the body, or ameliorate or alleviate the symptoms of the disease caused by the cancer. The treatment is considered therapeutic if there is a decrease in mortality and/or morbidity, or a decrease in disease burden manifest by reduced numbers of malignant cells in the body.

As used herein, and unless otherwise indicated, the term “preventing cancer” or “prevention of cancer” means to prevent the occurrence or recurrence of the disease state of cancer. As such, a treatment that impedes, inhibits, or interferes with metastasis, tumor growth, or cancer proliferation has preventive activity.

As used herein, and unless otherwise indicated, the term “managing” encompasses preventing the recurrence of cancer in a patient who had suffered from cancer, lengthening the time a patient who had suffered from cancer remains in remission, preventing the occurrence of cancer in patients at risk of suffering from cancer (e.g., patients who had been exposed to high amounts of radiation or carcinogenic materials, such as asbestos; patients infected with viruses associated with the occurrence of cancer, such as, but not limited to, HIV and Kaposi's sarcoma-associated herpesvirus; and patients with genetic predispositions to cancer, such as those suffering from Downs syndrome), and preventing the occurrence of malignant cancer in patients suffering from pre-malignant or non-malignant cancers.

As used herein, the phrases “therapeutically effective amount” and “prophylactically effective amount” refer to an amount that provides a therapeutic benefit in the treatment, prevention, or management of cancer. The specific amount that is therapeutically effective can be readily determined by ordinary medical practitioner, and may vary depending on factors known in the art, such as the type of cancer, the patient's history and age, the stage of cancer, the administration of other anti-cancer agents, including radiation therapy.

Methods of the invention can be used to treat and manage patients suffering from primary and metastatic cancer. They further encompass methods of treating patients who have been previously treated for cancer, as well as those who have not previously been treated for cancer. The invention encompasses first-line, second-line, third-line and further lines cancer treatments.

Cancers that can be treated and managed using methods of the invention include but are not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Additional examples of specific cancers include, but are not limited to: AIDS associated leukemia and adult T-cell leukemia lymphoma; anal carcinoma; astrocytoma; biliary tract cancer; cancer of the bladder, including bladder carcinoma; brain cancer, including glioblastomas and medulloblastomas; breast cancer, including breast carcinoma; cervical cancer; choriocarcinoma; colon cancer including colorectal carcinoma; endometrial cancer; esophageal cancer; Ewing's sarcoma; gastric cancer; gestational trophoblastic carcinoma; glioma; hairy cell leukemia; head and neck carcinoma; hematological neoplasms, including acute and chronic lymphocytic and myelogeneous leukemia; hepatocellular carcinoma; Kaposi's sarcoma; kidney cancer; multiple myeloma; intraepithelial neoplasms, including Bowen's disease and Paget's disease; liver cancer; lung cancer including small cell carcinoma; lymphomas, including Hodgkin's disease, lymphocytic lymphomas, non-Hodgkin's lymphoma, Burkitt's lymphoma, diffuse large cell lymphoma, follicular mixed lymphoma, and lymphoblastic lymphoma; lymphocytic leukemia; neuroblastomas; oral cancer, including squamous cell carcinoma; ovarian cancer, including those arising from epithelial cells, stromal cells, germ cells and mesenchymal cells; pancreatic cancer; prostate cancer; rectal cancer; sarcomas, including soft tissue sarcomas, leiomyosarcoma, rhabdomyosarcoma, liposcarcoma, fibrosarcoma, and osteosarcoma; skin cancer, including melanoma, Kaposi's sarcoma, basal cell cancer and squamous cell cancer; testicular cancer, including testicular carcinoma and germinal tumors (e.g., semicoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilm's tumor.

It would be readily apparent to one of ordinary skill in the art that the compounds of this invention (e.g., antisense oligonucleotides, siRNAs, and other agents described in section 5.1.3) of this invention can be combined with one or more of other anti-cancer therapies. The compounds of this invention can be administered simultaneously or sequentially with antineoplastic agents such as antimetabolites, alkylating agents, spindle poisons and/or intercalating agents, and proteins such as interferons.

Examples of particular second anti-cancer agents include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracycline; anthramycin; aromatase inhibitors; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; chlorodeoxyadenosine; cirolemycin; cisplatin; cladribine; corticosteroids; crisnatol mesylate; cyclophosphamide; cytarabine; cytosine arabinose; dacarbazine; dactinomycin; daunorubicin hydrochloride; decitabine; deoxyconformycin; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamnitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; leucovorin; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; myelopurine; navelbine; nitrosoureas camustine; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; progestins; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; taxane; tecogalan sodium; tegafur; teloxantrone hydrochloride; temoporfin; teniposide; teroxirone; testolactone; thiamiprine; thioguanine; thiotepa; tiazofurin; tirapazamine; topoisomerase inhibitors; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Still other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard second anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonennin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The determination of the identity and amount of second anti-cancer agent(s) for use in a method of the invention can be readily made by ordinarily skilled medical practitioners using standard techniques known in the art, and will vary depending on the type and severity of cancer being treated.

The compounds of this invention and second anti-cancer agents can be administered simultaneously or sequentially by the same or different routes of administration. The suitability of a particular route of administration employed for a particular compound will depend on the compound itself (e.g., whether it can be administered orally without decomposing prior to entering the blood stream) and the disease being treated. For example, treatment of tumors on the skin or on exposed mucosal tissue may be more effective if one or both active ingredients are administered topically, transdermally or mucosally (e.g., by nasal, sublingual, buccal, rectal, or vaginal administration). Treatment of tumors within the body, or prevention of cancers that may spread from one part of the body to another, may be more effective if one or both of the active ingredients are administered parenterally or orally. Similarly, parenteral administration may be preferred for the acute treatment of a disease, whereas transdermal or subcutaneous routes of administration may be employed for chronic treatment or prevention of a disease. Preferred routes of administration for the anti-cancer agents are known to those of ordinary skill in the art.

5.3 Pharmaceutical Compositions and Methods of Administrations

This invention encompasses pharmaceutical compositions comprising a compound that inhibits the synthesis or expression of tpt1 gene. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton, Pa. (1990).

5.3.1 Delivery of the Compounds of This Invention

Delivery of the compounds of this invention (e.g., antisense oligonucleotides, siRNAs, or other compounds described in section 5.1.3) into a patient can either be direct, i.e., the patient is directly exposed to the compounds of this invention or compound-carrying vector, or indirect, i.e., cells are first transformed with the compounds of this invention in vitro, then transplanted into the patient for cell replacement therapy. These two approaches are known as in vivo and ex vivo therapy, respectively.

In the case of in vivo therapy, the compounds of this invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector (U.S. Pat. No. 4,980,286, for example), by direct injection of naked DNA, by use of microparticle bombardment (for example, a gene gun; Biolistic®, DuPont), by coating with lipids or cell-surface receptors or transfecting agents, encapsulation in liposomes, microparticles, or microcapsules, by administering them in linkage to a peptide which is known to enter the cell or nucleus, or by administering them in linkage to a ligand subject to receptor-mediated endocytosis (Wu and Wu, J Biol. Chem. 262:4429-4432 (1987)), which can be used to target cell types specifically expressing the receptors. Further, the compounds of this invention can be targeted in vivo for cell specific uptake and expression, by targeting a specific receptor, as disclosed in, for example, WO 92/06180, WO 92/22635, WO92/20316, WO93/14188, and WO 93/20221. All of these references are incorporated herein by reference.

Ex vivo therapy involves transferring the compounds of this invention to cells in tissue culture by methods such as electroporation, lipofection, calcium phosphate mediated transfection, and viral infection. Usually, the method of transfer includes the transfer of a selectable marker to the cells. The cells are then placed under selection to isolate those cells that have taken up and are expressing the transferred compounds. Those cells are then delivered to a patient.

The compounds of this invention are introduced into a cell prior to administration in vivo of the resulting recombinant cell. Such introduction can be carried out by any method known in the art, including, but not limited to, transfection, electroporation, microinjection, infection with a viral vector containing the nucleic acid sequences, cell fusion, chromosome-mediated gene transfer, microcell-mediated gene transfer, and spheroplast fusion. Numerous techniques are known in the art for the introduction of foreign compounds into cells. Examples of such techniques are disclosed in: Loeffler et al., Meth. Enzymol. 217:599-618 (1993); and Cohen et al., Meth. Enzymol. 217:618-644 (1993); and Cline, Pharmac. Ther. 29:69-92 (1985), all of which are incorporated herein by reference. These techniques should provide for the stable transfer of the compounds of this invention to the cell, so that they are expressible by the cell and preferably heritable and expressible by its cell progeny.

The resulting recombinant cells can be delivered to a patient by various methods known in the art. Examples of the delivery methods include, but are not limited to, subcutaneous injection, skin graft, and intravenous injection.

5.3.2 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington 's Pharmaceutical Sciences, 18th ed., Mack Publishing, Easton, Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Disintegrants or lubiricants can be used in pharmaceutical compositions and dosage forms of the invention.

5.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention and second anti-cancer agents) disclosed herein can also be incorporated into the parenteral dosage forms of the invention.

5.3.4 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington 's Pharmaceutical Sciences, 16^{th} and 18^{th} eds., Mack Publishing, Easton, Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.4 Method of Identifying Genes Involved in Tumor Reversion

As discussed herein, this invention is based, in large part, on a discovery that tpt1 is not only involved in tumor reversion, but can affect that process. This invention also encompasses a method of identifying genes involved in tumor reversion. A particular method comprises: 1) determining a first set of genes that are differentially expressed (i.e., up-regulated or down-regulated) in a tumor cell as compared to its revertant or SIAH-1 transfected counterpart; 2) determining a second set of genes that are differentially expressed in a tumor cell of a different cell line as compared to its revertant or SIAH-1 transfected counterpart; and 3) identifying a gene that is common in both the first and second sets.

The expression of the genes in tumor cells and their revertants or SIAH-1 transfected counterparts can be followed using various methods known in the art for assessing the gene expression. These methods include, but are not limited to, differential display, MEGASORT®, Massively Parallel Signature Sequencing (“MPSS”), PCR, northern blot analysis and western blot analysis. In a specific method, the expression of the genes is compared using differential display, MEGASORT or MPSS.

The revertants can be generated by transfecting the tumor cell with H-1 parvovirus. It has been reported that H-1 parvovirus preferentially kills tumor cells while sparing their normal counterparts. Toolan, Nature 214: 1036 (1967). Based on this, tumor cells can be infected with H-1 parvovirus and surviving cells can be screened from the culture infected by the virus. Further, it has also been reported that SIAH-1 infected tumor cells display a reduced tumorigenicity. See, e.g., Roperch et al., Proc. Natl. Acad. Sci. USA 96: 8070-8073 (1999) and Bruzzoni-Giovanelli et al, Oncogene 18: 7101-7109 (1999), both of which are incorporated herein by reference in their entireties. Therefore, any tumor cell/revertant or tumor cell/SIAH-1 infected tumor cell pair can be used to test the differential expression of genes. Specific examples of such pairs include, but are not limited to, U937/US4.2, K562/KS6, BT20/BT20S, T47D/T47DS, MDA-MB231/MDA-MB231S, MCF7/MCF7-SIAH-1 and U937/U937-SIAH-1.

This method can be used to provide methods and compositions for the treatment, prevention and management of cancer. For example, when the method identifies a gene that is up-regulated in cancer cells, tumor reversion can be effected by suppressing the gene with a compound that inhibits its synthesis or expression.

### 6. EXAMPLES

Aspects of the invention are illustrated by the following non-limiting examples.

6.1 Selection and Characterization of Revertant Cells

The tumor cells K562, U937, T47D, MDA-MB231, BT20 and 184B5 were obtained from the American Type Culture Collection. The procedure involved a selection by H-1 parvovirus, which preferentially kills tumor cells while sparing the normal counterparts. Mousset et al., Nature 300: 537-539 (1982). Different concentrations of H-1 parvovirus were used to infect the tumor cells with a multiplicity of infection at 10-1,000 plaque forming units per cell. The medium was replaced once per week. The adherent tumor cell lines were isolated with cloning cylinders (Sigma) by using collagenase/dispase (Roche Diagnostics). Isolated revertants and the parental tumor cells were tested for their ability to form colonies in semisolid medium (agar-noble, Difco). The selected revertants are as follows: 1) KS-6 from myeloid leukemia K562 cell line; 2) BT20S from breast cancer, carcinoma of the mammary glands BT20 cell line; 3) T47DS from breast cancer, ductal carcinoma T47D cell line; and 4) MDA-MB231S from breast carcinoma MDA-MB231 cell line. In addition, to elucidate further information on tumor reversion, U937 and MCF7 cells were stably transfected with human SIAH-1 gene. The SIAH-1 transfected U937 and MCF7 cell lines are denoted as U937-SIAH-1 and MCF7-SIAH-1, respectively.

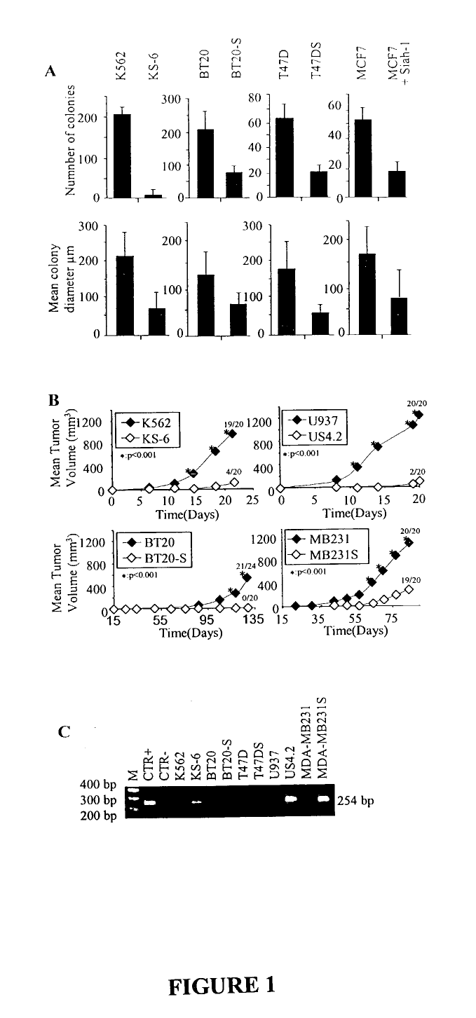

Characteristics of these selected revertants and SIAH-1 transfected cells are shown in FIG. 1A. FIG. 1A shows that the revertants of K562, BT20 and T47D cells, namely KS6, BT20S and T47DS, respectively, form significantly lower number of colonies with smaller mean diameter than their parent tumor cells. It is also shown in FIG. 1A that MCF-7 cells stably transfected by SIAH-1 exhibits similar characteristics as the selected revertants.

In vivo tumorigenicity tests were performed by infecting female scid/scid mice with 10⁷ cells per site. As shown in FIG. 1B, the revertants (KS-6, US4.2, BT20-S and MB231S) show significantly reduced tumorigenicity in scid/scid mice as compared with their parent tumor cells. From 20 rejections: KS-6 formed 4 tumors; US4.2 formed 2 tumors; BT20-S formed no tumors; and MB231S formed 19 tumors, but the tumor size was significantly lower that the ones formed from the parent tumor cells. MCF7 that was stably transfected with SIAH-1 gene did not grow in scid/scid mice.

To test whether the revertants continued to produce functional H-1 parvovirus after the initial infection, a 254 base pairs region of H-1 parvovirus DNA was amplified by PCR in various cells using the primers having the following sequences:

| | |
|---|---|
| 5′-CTAGCAACTCTGCTGAAGGAACTC-3′; and | (SEQ. ID NO.4) |
| 5′-TAGTGATGCTGTTGCTGTATCTGATG-3′. | (SEQ. ID NO.5) |

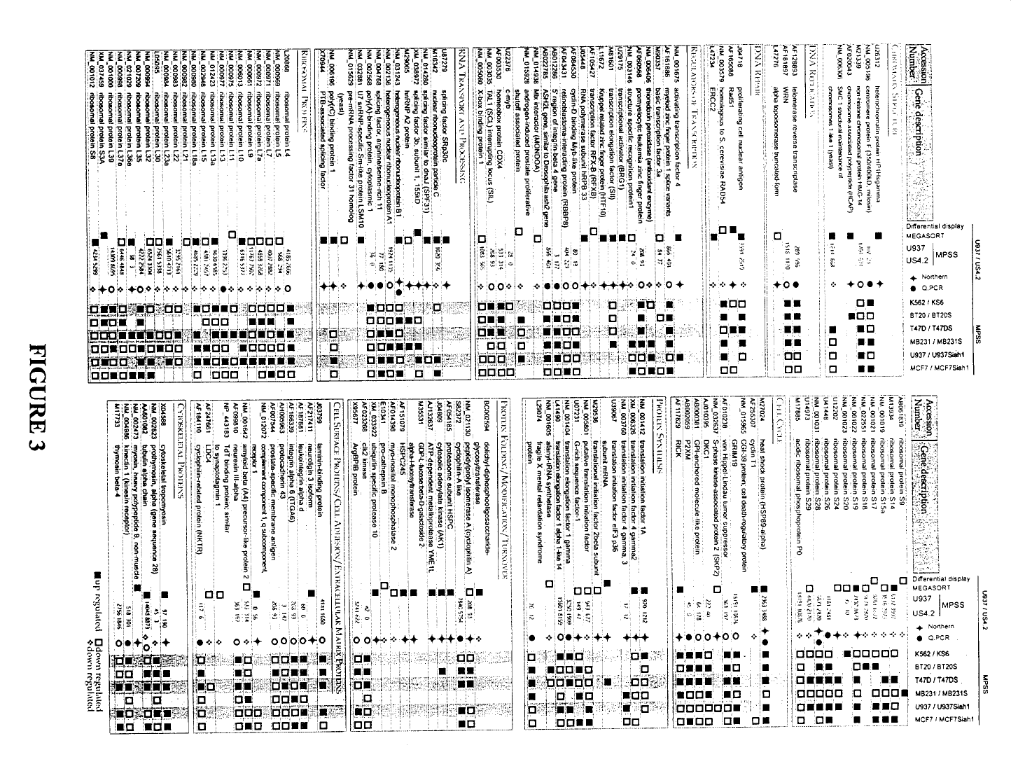

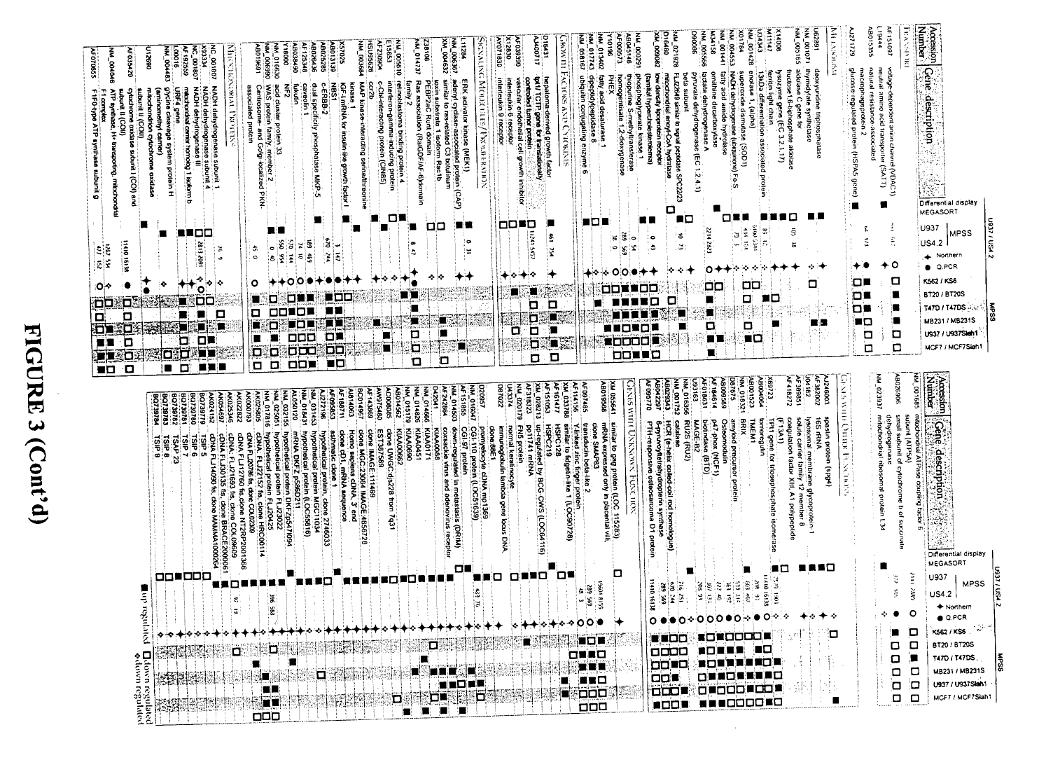

As shown in FIG. 3A, H-1 parvovirus DNA was detected in KS-6, US4.2 and MB231S, but not in BT20-S and T47DS.

6.2 Differential Gene Expression Analysis in Tumor Reversion

To identify the genes involved in tumor reversion, first the expression of all of the genes in U937 and its revertant US4.2 cells were analyzed. A differential display method, as disclosed in Liang et al., Science 257: 967-971 (1992), incorporated herein in its entirety by reference, was employed to test the expression. Further screenings were carried out using MEGASORT (Brenner et al., Proc. Natl. Acad. Sci. USA 97: 1665-1670 (2000), incorporated herein by reference), which-generates cDNA sequences as results, and Massively Parallel Signature Sequencing (“MPSS”, Brenner et al., Nature Biotech., 18: 630-634 (2000), also incorporated herein by reference), which generates signatures as results. MEGASORT and MPSS were performed at Lynx Therapeutics (Hayward, Calif.). From these screenings, two hundred sixty three genes were found to be differentially expressed in U937/US4.2 cell lines.

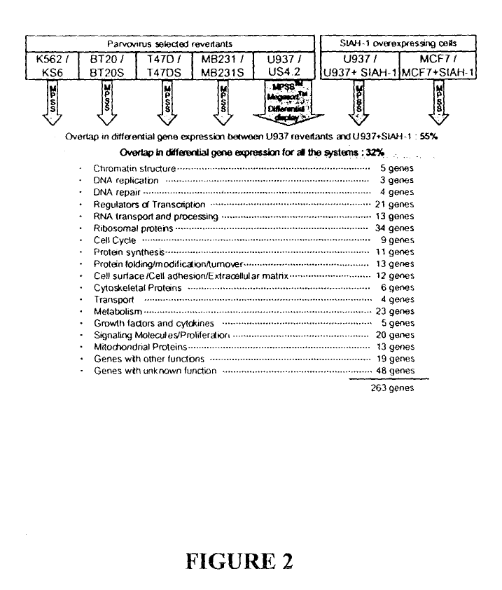

The expression of genes in six other tumor cell/revertant or tumor cell/SIAH-1 transfectant pairs were tested using MPSS. Specifically, the expression of the two hundred sixty three genes identified from the screening of U937/US4.2 cell lines were compared to the expression in the other six cell line pairs being tested in an effort to identify common effector genes involved in tumor reversion between different biological model systems. A schematic illustration of these procedures is shown in FIG. 2, and the results summarizing the expression of the two hundred sixty three genes are summarized in FIG. 3.

6.3 Differential Expression of tpt1/TCTP in Tumor Cells and Their Revertants

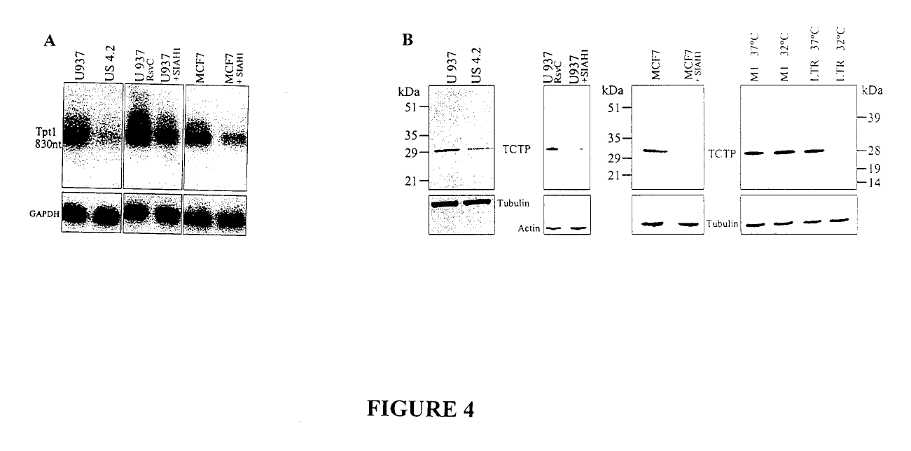

The expression of tpt1 gene was tested by northern blot in U937/US4.2, U937/U937-SIAH-1 and MCF7/MCF7-SIAH-1 cells. As shown in FIG. 4A, the expression of tpt1 is significantly reduced in the revertants or SIAH-1 transfected cells. In fact, tpt1 was the most differentially expressed in U937/US4.2 cell lines, with the signal detected 124 times in U937 versus only once in US4.2, when subjected to MEGASORT screening. As an indicator for equal loading, the expression of GAPDH was monitored. As shown in FIG. 4A, the band intensities obtained for GAPDH in the revertants or SIAH-1 transfected cells were substantially the same as the parent tumor cells.

The expression of TCTP (the product of tpt1 gene) was also monitored in revertants or SIAH-1 transfected cells as compared with the parent tumor cells by using western blot analysis. As shown in FIG. 4B, the revertant US4.2 cells and U937 and MCF7 cells transfected with SIAH-1 all exhibited a substantially reduced expression of TCTP. Actin and tubulin were included as a control to ensure equal loading. It is notable that LTR6 system, after activation of wild-type p53 function, showed a drastic decrease of TCTP level. As the LTR6 system is known to be a good indicator of cell apoptosis, this result indicates that TCTP is involved in cell apoptosis.

6.4 Inhibition of tpt1 Expression by tpt1 Antisense cDNA

To mimic the natural phenomenon of tpt1 inhibition during tumor reversion, tpt1 expression in tumor cells was inhibited using antisense technique. The cDNA corresponding to the coding region of tpt1 was cloned in an inverted way in pBK-RSV® from Stratagene. U937 cells were transfected with tpt1 antisense cDNA using Lipofectin® from Invitrogen, followed by selection with 1.5 mg/ml G418.

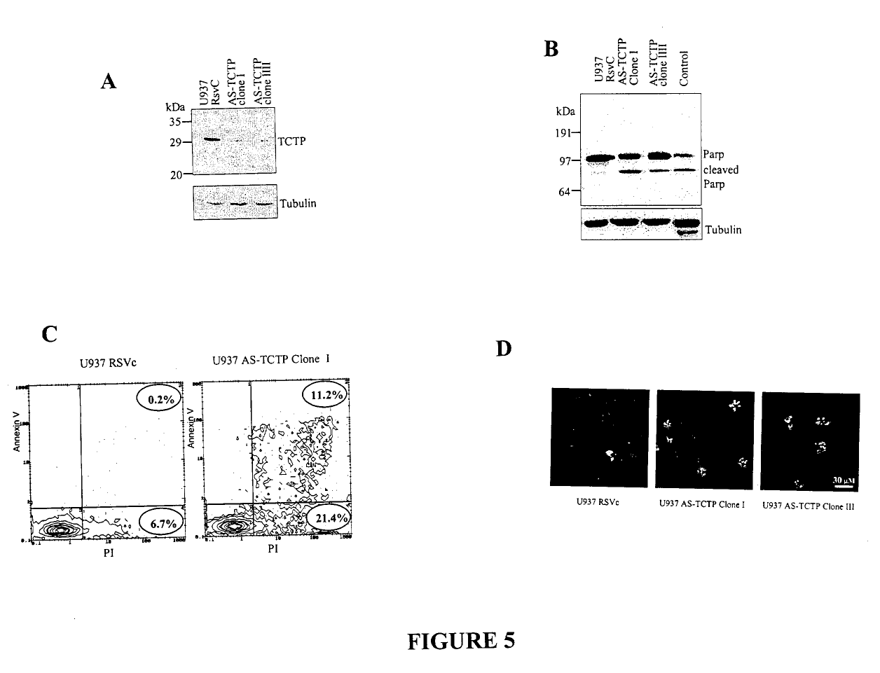

As illustrated in FIG. 5A, U937 cells transfected with tpt1 antisense cDNA showed little expression of TCTP, whereas the same cells transfected with vector alone showed significant level of TCTP expression. Tubulin was included to ensure equal loading.

To test whether this induced inhibition of tpt1 expression would affect cell apoptosis, the rate of apoptosis was assessed by using Poly ADP-Ribose Polymerase (PARP) cleavage test. A specific anti-PARP antibody was used to visualize the location of PARP. As shown in FIG. 5B, while little cleavage occurred in U937 cells transfected with vector alone, increased PARP cleavage was observed for U937 cells transfected with tpt1 antisense cDNA. This indicates that inhibition of tpt1 expression yields an increased in cell apotosis. To confirm this, two additional tests, namely annexin V and TUNEL assays, were performed. The results were in accord with what was obtained from PARP cleavage test, showing an increase in annexin V content in U937 cells transfected with tpt1 antisense cDNA (FIG. 5C) and in TUNEL positive cells (FIG. 5D). The increase in cell apoptosis in these tests were consistently reproducible.

6.5 Tumorigenicity of tpt1 Antisense cDNA Transfected U937 Cells

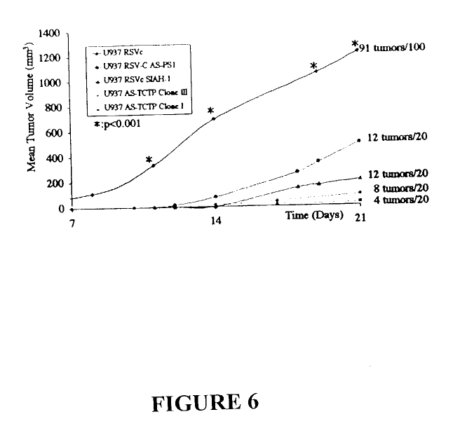

Scid/scid mice were injected with 10⁷ cells per site each of: U937 cells; U937 cells transfected with antisense PS-1; U937 cells transfected with SIAH-1; and U937 cells transfected with antisense tpt1 (clones I and III comprising SEQ. ID NO. 6). PS-1 and SIAH-1 have previously been reported to reduce tumorigenicity. As shown in FIG. 6, the reduction of tumorigenicity in both U937 cells transfected with antisense tpt1 was more profound than other cells tested. Moreover, the injection of U937 cells transfected with antisense tpt1 resulted in significantly smaller tumor sizes than either U937 cells, PS-1 transfected U937 cells or SIAH-1 transfected U937 cells.

6.6 The Expression of TCTP in Various Tumor Cells

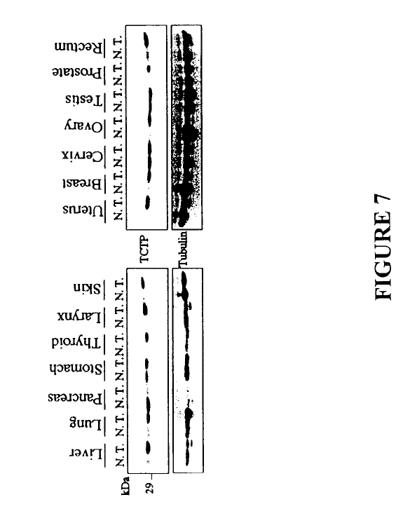

The expression of TCTP in various tumor cells was tested by using western blot analyses of normal and tumor cells from different organs. TCTP was visualized using specific TCTP antibodies. As shown in FIG. 7, the expression of TCTP was higher in most tumor cells when compared with their normal counterpart. This shows that TCTP is up-regulated in most cancer cells.

6.7 Knock-down of tpt1 by siRNA

The tpt1 was knocked-down in MCF7 and T47D cells by using siRNA. The procedures as described in Elbashir et al., Nature 411: 494-498 (2001), which is incorporated herein in its entirety by reference, were followed. RNA duplexes with 5′ dTdT overhang directed against the following sequences of tpt1 mRNA were synthesized (Dharmacon Research, Lafayette, Colo.):

| | |
|---|---|
| 5′-AAGGTACCGAAAGCACAGTAA-3′; and | (SEQ. ID NO.1) |
| 5′-AACCATCACCTGCAGGAAACA-3′. | (SEQ. ID NO.2) |

Mouse trt/TCTP siRNA duplex of the following sequence was used as a control:

| | |
|---|---|
| 5′-AACCATCACTTACAAGAAACC-3′. | (SEQ. ID NO.3) |

The cells with knocked-down tpt1 were then subjected to 3D reconstituted basement membrane matrigel cultures for further investigation.

MCF7 and T47D cells were transfected with 1 nM siRNA by using Oligofectamine® from Invitrogen. Cells were further incubated for 3 days. Cells were then detached, counted, and mixed 1:1 with Matrigel® (Becton Dickinson). The resulting cell concentration was 2×10⁵ cells per ml, and the matrigel concentration was 6.25 mg/ml. Cells were stained with anti-E-cadherin antibodies (Transduction Laboratories, Lexington, Ky.), and nuclei were stained with propidium iodide and analyzed by confocal microscopy.

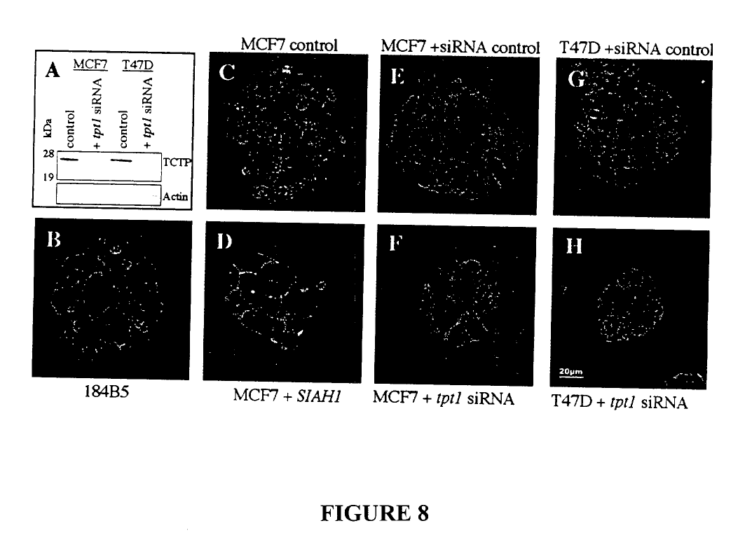

FIG. 8A shows the western blot analysis of TCTP expression in MCF7 and T47D cells with and without the transfection with tpt1 siRNA. The results confirm that tpt1 in these cells were properly knocked down form the above procedures. Actin was included to ensure equal loading.

As shown in FIGS. 8B through 8H, MCF7 and T47D cells transfected with tpt1 siRNA (FIGS. 8F and 8H) showed a drastic difference in the matrigel from MCF7 (FIG. 8C), MCF7 transfected with mouse trt siRNA (FIG. 8E), or TD47D transfected with mouse trt siRNA (FIG. 8G). Moreover, tpt1 siRNA transfected cells formed structures that reflected the growth of 184B5 cells (FIG. 8B), which are non-tumorigenic cells included as a normal control. MCF7 cells stably transfected with SIAH-1 (FIG. 8D), which has a reduced TCTP expression, also exhibited similar characteristics as MCF7 and T47D cells transfected with tpt1 siRNA.

All of the patents, patent applications and publications referred to in this application are incorporated herein in their entireties. Moreover, citation or identification of any reference in this application is not an admission that such reference is available as prior art to this invention. The full scope of the invention is better understood with reference to the appended claims.

What is claimed is:

1. A method of suppressing growth of a cancer cell, comprising contacting the cell with a compound that inhibits the synthesis or expression of tpt1 in an amount sufficient to cause such inhibition.

2. A method of suppressing growth of a cancer cell, comprising contacting the cell with a compound that has a sequence complementary to at least part of tpt1 mRNA.

3. The method of claim 2, wherein the compound is an oligonucleotide antisense to tpt1 mRNA.

4. The method of claim 3, wherein the oligonucleotide is a cDNA that transcribes into an RNA having a sequence complementary to at least part of the tpt1 mRNA.

5. The method of claim 2, wherein the compound is a tpt1 siRNA.

6. The method of claim 5, wherein the siRNA has a sequence corresponding to SEQ. ID NO. 1 or SEQ. ID NO. 2.

7. The method of claim 2, wherein the inhibition reduces the amount of TCTP in the cancer cell by about 20% or more.

8. The method of claim 7, wherein the inhibition reduces the amount of TCTP in the cancer cell by about 50% or more.

9. The method of claim 8, the inhibition reduces the amount of TCTP in the cancer cell by about 70% or more.

10. The method of claim 2, wherein the growth suppression is apoptosis.

11. The method of claim 2, wherein the growth suppression is reversion.

12. A method of treating, preventing or managing cancer comprising administering to a patient in need of such treatment, prevention or management a therapeutically or prophylactically effective amount of a compound that inhibits the synthesis or expression of tpt1.

13. A method of treating, preventing or managing cancer comprising administering to a patient in need of such treatment, prevention or management a therapeutically or prophylactically effective amount of a compound that has a sequence complementary to at least part of tpt1 mRNA.

14. The method of claim 13, wherein the compound is an oligonucleotide anti-sense to tpt1 mRNA.

15. The method of claim 14, wherein the oligonucleotide is a cDNA that transcribes into an RNA having a sequence complementary to at least part of the tpt1 mRNA.

16. The method of claim 13, wherein the compound is a tpt1 siRNA.

17. The method of claim 16, wherein the siRNA has a sequence corresponding to SEQ. ID NO. 1 or SEQ. ID NO. 2.

18. A pharmaceutical composition comprising a compound that inhibits the synthesis or expression of tpt1.

19. A pharmaceutical composition comprising a compound that has a sequence complementary to at least part of tpt1 mRNA.

20. The pharmaceutical composition of claim 19, wherein the compound is an oligonucleotide antisense to tpt1 mRNA.

21. The pharmaceutical composition of claim 20, wherein the oligonucleotide is a cDNA that transcribes into an RNA having a sequence complementary to at least part of the tpt1 mRNA.

22. The pharmaceutical composition of claim 19, wherein the compound is a tpt1 siRNA.

23. The pharmaceutical composition of claim 22, wherein the siRNA has a sequence corresponding to SEQ. ID NO. 1 or SEQ. ID NO. 2.

24. A method of identifying genes involved in tumor reversion comprising:
1) determining a first set of genes that are differentially expressed in a tumor cell as compared to its revertant or SIAH-1 transfected counterpart;
2) determining a second set of genes that are differentially expressed in a tumor cell of a different cell line as compared to its revertant or SIAH-1 transfected counterpart; and
3) identifying a gene that is common in both the first and second sets.

25. The method of claim 24, wherein the revertant of the tumor cell is generated by transfecting the tumor cell with H-1 parvovirus.

26. The method of claim 24, wherein the tumor cell and its revertant or SIAH-1 transfected counter part are U937/US4.2, K562/KS6, BT20/BT20S, T47D/T47DS, MDA-MB231/MDA-MB231S, MCF7/MCF7-SIAH-1 or U937/U937-SIAH-1.

27. A method of identifying a chemotherapeutic agent, which comprises: 1) identifying a gene that is up-regulated in a cancer cell according to the method of claim 24; and 2) identifying a compound that inhibits the synthesis or expression of the gene.

28. The method of claim 27, wherein the compound is an oligonucleotide antisense to mRNA of said gene.

29. The method of claim 28, wherein the oligonucleotide is a cDNA that transcribes into an RNA having a sequence complementary to at least part of the mRNA.

30. The method of claim 27, wherein the compound is an siRNA having a sequence complementary to the mRNA.

31. A method of treating, preventing or managing cancer in a patient, which comprises: 1) identifying a gene that is up-regulated in a cancer cell according to the method of claim 24; 2) identifying a compound that inhibits the synthesis or expression of the gene; and 3) administering a therapeutically or prophylactically effective amount of the compound to a patient in need of such treatment, prevention or management.


SEQUENCE LISTING (6 sequences)

SEQ ID NO: 1 - DNA, 21 bp, Homo sapiens
aaggtaccgaaagcacagtaa

SEQ ID NO: 2 - DNA, 21 bp, Homo sapiens
aaccatcacctgcaggaaaca

SEQ ID NO: 3 - DNA, 21 bp, Mouse
aaccatcacttacaagaaacc

SEQ ID NO: 4 - DNA, 24 bp, Artificial Sequence
  Synthesized Primer
ctagcaactctgctgaaggaactc

SEQ ID NO: 5 - DNA, 26 bp, Artificial Sequence
  Synthesized Primer
tagtgatgctgttgctgtatctgatg

SEQ ID NO: 6 - DNA, 519 bp, Homo sapiens
ttaacatttttccatttctaaaccatccttaaagaaaatcatatatggggtcacaccatcctcacggtagtccaatagagcaaccatgccatctggattcatgttttcaccaataaagaactggtagtttttgaaattagcaaggatgtgcttgatttgttctgcagcccctgtcataaaaggttttactctttctggtctctgttcttcaagtttccctttgattgatttcatgtaatctttgatgtacttcttgtaggcttcttttgtgaaacttgtttcctgcaggtgatggttcatgacaatatcgacaccagtgattactgtgctttcggtaccttcgccctcggggccttcagcggaggcatttccaccaatgagcgagtcatcaatgttaccttctgtcctactgaccatcttcccctccacctccaggcacaacccgtccgcgatctcccggatcttgtagatgtcggagaacatctcatcgtggctgatgaggtcccggtagataatcat